(19) United States
(12) Patent Application Publication
(10) Pub. No.: US 2011/0244031 A1
(43) Pub. Date: Oct. 6, 2011

(54) POROUS TABLETS AS CARRIERS FOR LIQUID FORMULATIONS

(75) Inventors: Nikolaj SKAK, Virum (DK); Bent HOJGAARD, Allerod (DK)

(73) Assignee: LifeCycle Pharma A/S, Horsholm (DK)

(21) Appl. No.: 13/077,290

(22) Filed: Mar. 31, 2011

Related U.S. Application Data
(60) Provisional application No. 61/322,086, filed on Apr. 8, 2010.

(30) Foreign Application Priority Data
Mar. 31, 2010 (DK) 2010-00277

(51) Int. Cl. A61K 9/48; B29C 43/02; A61K 9/00; A61K 9/14; A61K 36/899; A61K 31/202; A61K 47/04

(52) U.S. Cl. 424/451; 264/115; 424/400; 424/489; 424/750; 514/560; 514/770

(57) ABSTRACT

A tablet composition, which in an easy, flexible and reproducible manner can be loaded with a relatively high amount of a pharmaceutically acceptable oily substance, may be produced in large-scale batches and stored until use; and tablets loaded with a pharmaceutically acceptable oily substance as well as a method for the preparation thereof. The tablet composition comprises a release enhancing agent and provides high and/or consistent release of the pharmaceutically acceptable oily substance, in particular release of a pharmaceutically acceptable, but substantially water-insoluble, oily substance.

The present invention relates to a novel inert carrier composition, preferably in the form of a tablet product, which in an easy, flexible and reproducible manner can be loaded with a relatively high amount of a pharmaceutically acceptable oily substance and/or a pharmaceutically active substance. The novel composition is pre-deposited with a release enhancing agent and may be produced in large-scale batches and stored until use and each batch or sub-batch may be loaded with the same or different pharmaceutically acceptable oily substances. The invention also provides tablets that have been loaded with such a pharmaceutically acceptable oily substance as well as a method for the preparation thereof. The composition of the present invention provides high and/or consistent release of the pharmaceutically acceptable oily substance, in particular a pharmaceutically acceptable oily substance which is substantially water insoluble.

### BACKGROUND OF THE INVENTION

Many drug substances have and it is expected that many of the future drug substances will have undesired properties especially with respect to e.g. water solubility and to oral bioavailability. Therefore, novel technologies, which enable especially therapeutically and/or prophylactically active substances to be delivered to the body in a relatively easy manner and at the same time enables the desired therapeutic and/or prophylactic response, is highly needed.

In the pharmaceutical area it is common to prepare pharmaceutical compositions comprising one or more active substances and various excipients. One reason for preparing such pharmaceutical compositions is to manipulate the availability of the active compound after ingestion of the pharmaceutical composition.

For the preparation of pharmaceutical composition for oral administering the active substances are often incorporated into an agglomerated preparation in order to provide the active compounds in a form that may be pressed into tablets or filled into capsules.

One commonly used technique for granulation is a wet granulation, where a mixture of powders including the active compound is mixed with a liquid, usually an aqueous liquid, under mechanical influence for the preparation of granules. Usually the granules prepared by wet granulation are dried before use.

Melt agglomeration and controlled agglomeration are techniques for agglomeration of an active compound, essentially performed by melting a pharmaceutical acceptable vehicle such as an oil or an oily-like material, dissolution or dispersion of one or more active compounds in the melted vehicle and deposition of the thus prepared mixture on a particulate material, the filler, and subsequently the particles adhere to each other and form agglomerates.

WO 03/004001 discloses the novel technique of controlled agglomeration by which it is possible to load a particulate material with a relatively high amount of an oil or an oily-like material. The technique is based on a process that involves spraying of a carrier composition containing the oil or oily-like material onto a particulate material. The process conditions enable the particulate material to be loaded with a relatively high amount of the oil or oily-like material. Normally, the process involves heating of the carrier composition and maintaining the temperature of the carrier composition during application. As the application is performed by spraying, strict temperature control of the spraying equipment is a requirement in order to avoid problems relating to clotting of the spray nozzle etc.

WO 2006/000227A2 discloses the preparation of a tablet solely containing inert pharmaceutically acceptable excipients (although in some cases it may be suitable also to incorporate an active substance therein) and when the tablet is subjected to a pharmaceutically acceptable liquid formulation e.g. containing the active substance, the tablet will due to its porosity absorb the liquid formulation. This loading of an inert tablet takes place within a relatively short period of time and is reproducible, i.e. the same amount of liquid formulation is absorbed when the same type and size of tablet and liquid formulation is used.

### SUMMARY OF THE INVENTION

The present invention relates to a pharmaceutically inert composition, i.e. a loadable solid porous composition comprising a porous silicium dioxide (silicon dioxide) and a release enhancing agent, in particular a loadable solid porous composition consisting essentially of porous silicium dioxide pre-deposited with a release enhancing agent.

The inventors have found that the release tends to decrease over time of a pharmaceutically acceptable oily substance and/or a pharmaceutically active substance, preferably in liquid form, loaded into an inert porous tablet such as those disclosed in WO 2006/000227A2. Such oily substances may be oils or oil derivatives, emulsions, micro-emulsions (SMEDDS), nano-emulsions (SNEDDS), which are substantially water insoluble (or water immiscible) or form an emulsion when dissolved in water. For instance, full release may not be or is not obtained as the initial release of e.g. corn oil is approximately 85% in water. The release of corn oil drops to approximately 40% in two weeks and a further decline in release to approximately 35% in a month.

In order to obtain a consistent release, and preferably a high release, that is maintained over time the inventors have developed a loadable solid porous composition, typically a compressed tablet, comprising a porous silicium dioxide, such as an aluminum silicate, e.g. magnesium aluminum metasilicate, and a release enhancing agent, such as a polymer or an inorganic aqueous hydrogen phosphate, e.g. PEG or KH₂PO₄, or mixtures thereof. This loadable composition is able to absorb the pharmaceutically acceptable oily substance, such as an edible oil or fat, e.g. a vegetable oil or fat or an animal oil or fat, and release the pharmaceutically acceptable oily substance in a high and consistent manner and maintain the release over time.

A liquid loadable tablet without a release enhancing agent, such as a polymer or an inorganic aqueous hydrogen phosphate, e.g. PEG6000 or KH₂PO₄, or mixtures thereof, increase its disintegration rate over time (experiments show an increase in disintegration rate to 35 minutes in 7 days).

Furthermore, by including a disintegrant, such as croscarmellose sodium in the loadable solid porous composition comprising a porous silicium dioxide, such as an aluminum silicate, and a release enhancing agent, the inventors obtained a solid composition, which when compressed into a tablet, showed a fast disintegration rate which was maintained for the solid porous composition comprising a porous silicium dioxide, such as an aluminum silicate, and a release enhancing agent regardless of the type of release enhancing agent, such as a polymer or an inorganic aqueous hydrogen phosphate. In particular, fast disintegration is maintained even at low concentrations of the disintegrant.

The loadable as well as the loaded solid porous composition comprising a porous silicium dioxide, such as an aluminum silicate, and a release enhancing agent, and optionally a disintegrant are prepared by methods as described herein.

Furthermore, the inventors have realized that by using a release enhancing agent, such as a polymer or an inorganic aqueous hydrogen phosphate, e.g. PEG6000 or KH₂PO₄, or mixtures thereof, for treating a porous silicium dioxide, the porous silicium dioxide, such as granules of porous silicium dioxide, e.g. Neusilin, will increase the ability to release a pharmaceutically acceptable oily substance from such a granule and also maintain the ability to release a pharmaceutically acceptable oily substance from the granules.

Without being bound by this theory, the decrease in oily substance release observed with liquid loadable tablets could relate to an interaction of oil with the silonyl groups of the magnesium aluminometasilicate sold under the trade name Neusilin® by Fuji Chemicals Co. (www.neusilin.com), lipid ordering on Neusilin surfaces, inversion of Neusilin structure facilitated by oil/water contact, inversion of the Neusilin structure facilitated by structural tension from tablet compaction process.

### DETAILED DESCRIPTION OF THE INVENTION

As used herein, the term “pre-deposited” reflects that a release enhancing agent has been contacted with, and deposited onto, the porous silicium dioxide used in the composition of the present invention simultaneously with or prior to subjecting the silicium dioxide material to further processing into, e.g. granules, tablets or other forms, including inert loadable porous tablets capable of absorbing liquid substances and, subsequently, releasing the absorbed substances fully or partly.

The present invention relates to a loadable solid porous composition comprising a porous silicium dioxide and a release enhancing agent. The composition is prepared from a solid porous granulate comprising a mixture of porous silicium dioxide and a release enhancing agent, such granulate or granules also being an aspect of the present invention. Typically, the porous silicium dioxide is pre-deposited with the release enhancing agent.

The granulate comprising a porous silicium dioxide (silicon dioxide) and a release enhancing agent may be compacted, such as compressed or molded to a tablet that has a suitable hardness, such as a hardness of 20 N or more, typically, the tablet has a hardness of 25 N or more, about 30 N or more, about 35 N or more, about 40 N or more, about 45 N or more, about 50 N or more, about 60 N or more, about 70 N or more, about 90 N or more, about 100 N or more, about 150 N or more or about 200 N. Typically from about 30 N to about 150 N, such as 30 N to 100 N.

In another embodiment the granulate comprising a porous silicium dioxide and a release enhancing agent is used as is for loading a pharmaceutically acceptable oily substance. The granulate may be saturated with the pharmaceutically acceptable oily substance and filled into a suitable container means, such as a capsule, however, typically the pharmaceutically acceptable oily substance is loaded to about 95% of the loading capacity, to avoid batch-to-batch variations, such as about 90%, about 80%, about 70%, preferably the oily substance is loaded from 80% to 100% of the loading capacity.

The term “a pharmaceutically acceptable oily substance” as defined herein means a natural or synthetic oil or oil derivative, emulsions, water-in-oil, oil-in-water, wherein such oils or emulsions may further comprise a dissolved active pharmaceutical ingredient selected from small organic molecules, steroids, amino acids, peptides, proteins, RNA, DNA. Also intended covered is mixtures of oils with other ingredients, e.g. co-solvents, surfactants, co-surfactants, such as self-micro emulsifying drug delivery systems (SMEDDS) or nano self emulsifying drug delivery systems (NSEDDS), which upon contact with water forms an emulsion.

The term “a porous silicium dioxide” or “porous silicate” as defined herein means one or more silicates which can be divided in the following groups:
Swelling clays of the smectite type e.g. bentonite, veegum, laponite.
Hydrous aluminium silicates or alkaline earths. Neusilin belongs to this group and is based on synthetic polymerisation (magnesium aluminium metasilicate).
Silicon dioxides are subdivided into porous and nonporous silicas
   Nonporous colloidal silicas e.g. Aerosil (fumed silicas)
   Porous silicas gels e.g. Syloid, Porasil, Lichrosorp
   Others e.g. precipitated silicate, Zeopharm 5170, Zeopharm 600, and Rxcipients GL200, Huber Corporation, N.J., or Aeroperl 300

Accordingly, embodiments of the porous silicate to be used in a loadable or loaded solid porous composition of the present invention is selected from sodium silicate, potassium silicate, magnesium silicate, calcium silicate, including synthetic calcium silicate such as, e.g., Hubersorp, zink silicate, aluminum silicate, sodium aluminosilicate such as, e.g., Zeolex, magnesium aluminum silicate, magnesium aluminum metasilicate, aluminium metasilicate, Neusilin SG2 and Neusilin US2 and mixtures thereof. In a further embodiment the porous silicate is selected from precipitated silicate, sodium silicate, potassium silicate, magnesium silicate, calcium silicate, synthetic calcium silicate, zink silicate, aluminum silicate, such as sodium aluminosilicate, magnesium aluminum silicate, magnesium aluminum metasilicate and aluminium metasilicate; and mixtures thereof.

The aluminum silicate is a highly porous material having a typical average pore size of 30 to 80, such as 50-60 angstrom and a surface area of from 250 to 400 m²/g, such as about 300 m²/g. The composition of the present invention typically has a porosity of 30% v/v or more, which is necessary for absorption of a suitable amount of a pharmaceutically acceptable oily substance.

In further embodiments the porosity is 40% v/v or more, 50% v/v or more, 60% v/v or more, 70% v/v or more, 80% v/v or more, or 90% v/v or more. The porosity is measured on the porous silicium dioxide, such as Neusilin, and then it is calculated how much porous silicium dioxide and the release enhancing agent, such as KH2PO₄, utilize of the porosity.

The porosity of the granules or tablets before loading is calculated on basis of the density of the granule or tablet ρₜ and the “true density” ρₛ of the ingredients. The porosity ε of the granule or tablet is calculated according to the Equation 1.

$$ɛ = 1 - \frac{{ρ}_{t}}{{ρ}_{s}} \qquad Equation 1$$

The density of the granule or tablet is based on the ratio between weight and volume of the granule or tablet. The “true density” of the ingredients is based on the gas pycnometric density determined in helium using Micromeritics Accupyc 1330.

The maximum loading capacity of corn oil on weight basis is calculated according to Equation 2.

$$loading capcity w \text{/} w \% = \frac{ɛ}{ɛ + ( 1 - ɛ ) \frac{{ρ}_{s}}{{ρ}_{l}}} 100 \qquad Equation 2$$

The density of corn oil, ρ₁=0.92 g/cm³

In a further embodiment the composition of the present invention the porous silicium dioxide is typically present in a concentration of about 20% w/w or more. It is apparent that the higher porosity desired the higher the concentration of the porous silicium dioxide, thus in further embodiments of the composition of the present invention the porous silicium dioxide is present in a concentration of about 25% w/w or more, about 30% w/w or more, about 35% w/w or more, about 40% w/w or more, about 45% w/w or more, about 50 w/w or more, about 60% w/w or more, about 70% or more, about 80% or more, about 90% or more, about 95% or more, or about 98% or more, in the unloaded composition.

As described above the aluminum silicate typically has an average pore size of 30 to 80, such as 50-60 angstrom and a surface area of from 250 to 400 m²/g, such as about 300 m²/g. In an embodiment the aluminum silicate is selected from magnesium aluminum metasilicate, magnesium aluminum silicate, and aluminium metasilicate, and mixtures thereof. Typical examples of aluminum silicates are Neusilin SG2, and

Neusilin US2, and mixtures thereof, in particular Al₂O₃.MgO.ySiO₂.xH₂O, wherein y is from 1.5-2, and x is 1-10, preferred is magnesium aluminum metasilicate, e.g. Al₂O₃.MgO.2SiO₂.5H₂O.

In order to obtain a solid porous composition of the present invention that will release a consistent amount of a pharmaceutically acceptable oily substance, and preferably also a high amount of the pharmaceutically acceptable oily substance, when such oily substance is loaded into the composition, such as a tablet or granule, it is necessary to treat the porous silicium dioxide with a release enhancing agent before loading the oily substance. The release enhancing agent is not a major component of the composition of the present invention and the purpose of using a release enhancing agent is to obtain granulates of porous silicium dioxide that are capable of absorbing and releasing the pharmaceutically acceptable oily substance in a high and consistent manner. The porous silicium dioxide may be treated with a surplus of the release enhancing agent to produce the treated porous silicium dioxide, and the release enhancing agent in the composition after treatment is typically present in a concentration of about 2% w/w or more, such as about 5% w/w or more, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 60% w/w or more or about 70% or more (based on the total weight of the composition before loading), such as from 5% to 70% w/w, e.g. from 35% to 50% w/w.

The release enhancing agent modifies the surface of the porous silicium dioxide to such an extent that the following release of an absorbed pharmaceutically acceptable oily substance, in particular, a water immiscible, water insoluble or substantially water insoluble oily substance, is high and consistent, which is of importance when preparing pharmaceutical or nutritional compositions for administration to a mammal, such as a human subject. In an embodiment the release enhancing agent is selected from a polymer. In another embodiment the release enhancing agent is selected from an inorganic aqueous hydrogen phosphate. In a further embodiment the release enhancing agent is selected from an inorganic salt, such as an alkaline salt, e.g. NaCl.

In a further embodiment of the present invention the polymer is selected from polyethylene glycol, poloxamer, polyethylene oxide, an alkyl cellulose, or polyvinyl alcohol (PVA), polyvinyl acetate phthalate, polyvinyl acetate or mixtures thereof. Mixtures also includes mixtures of PEGs, mixtures of poloxamers, mixtures of alkyl celluloses, and mixtures of PVAs.

Examples of poloxamers include Poloxamer 188, Poloxamer 237, Poloxamer 338 or Poloxamer 407 or other block copolymers of ethylene oxide and propylene oxide such as the Pluronic® and/or Tetronic® series, or mixtures thereof. Suitable block copolymers of the Pluronic® series include polymers having a molecular weight of about 3,000 or more such as, e.g. from about 4,000 to about 20,000 and/or a viscosity (Brookfield) from about 200 to about 4,000 cps such as, e.g., from about 250 to about 3,000 cps. Suitable examples include Pluronic® F38, P65, P68LF, P75, F77, P84, P85, F87, F88, F98, P103, P104, P105, F108, P123, F123, F127, 10R8, 17R8, 25R5, 25R8 etc. Suitable block copolymers of the Tetronic® series include polymers having a molecular weight of about 8,000 or more such as, e.g., from about 9,000 to about 35,000 and/or a viscosity (Brookfield) of from about 500 to about 45,000 cps such as, e.g., from about 600 to about 40,000. The viscosities given above are determined at 60 cc for substances that are pastes at room temperature and at 77° C. for substances that are solids at room temperature.

Examples of alkyl celluloses include hydroxylpropyl methyl cellulose (HPMC), hydroxy propyl cellulose (HPC), hydroxy propyl methyl cellulose acetate succinate (HPMCAS), hydroxypropyl methylcellulose phthalat (HPMCP)

Examples of polyethylene glycols (PEG)s include PEG having an average molecular weight in a range of from about 400 to about 100,000, such as from about 400 to about 35,000 such as, e.g., from about 800 to about 35,000, from about 1,000 to about 35,000 such as, e.g., polyethylene glycol 1,000, polyethylene glycol 2,000, polyethylene glycol 3,000, polyethylene glycol 4,000, polyethylene glycol 5,000, polyethylene glycol 6000, polyethylene glycol 7,000, polyethylene glycol 8,000, polyethylene glycol 9,000 polyethylene glycol 10,000, polyethylene glycol 15,000, polyethylene glycol 20,000, or polyethylene glycol 35,000; and mixtures thereof. In a further embodiment PEG is PEG having an average molecular weight in a range of from about 1000 to about 20,000, e.g. PEG6000.

In a further embodiment of the present invention the release enhancing agent is an inorganic salt, such as NaCl. In a further embodiment of the present invention the release enhancing agent is an inorganic aqueous hydrogen phosphate selected from alkali, such as sodium, potassium, and magnesium hydrogen phosphate, or sodium, potassium, and magnesium dihydrogen phosphate, phosphoric acid or phosphorous acid. In a particular embodiment the inorganic aqueous hydrogen phosphate is potassium dihydrogen phosphate (KH₂PO₄). In some of the experiments wherein the porous silicium dioxide, in particular the magnesium aluminum metasilicate, was treated with KH₂PO₄, such as surplus of KH₂PO₄, the concentration of KH₂PO₄ was most effective in the molar concentration from 0.1 M to 10 M, such as from 0.1 M to 2 M, e.g. 0.1 M to 0.5 M, 0.33 M to 1 M, or even at 0.1 M, 0.33 M, 0.5 M or 1 M.

The release enhancing agent as used herein may be a mixture of agents, and in a particular embodiment is selected from a mixture of a polymer and an inorganic salt or a polymer and an inorganic aqueous hydrogen phosphate. As an example the release enhancing agent is selected from a mixture of PEG and KH₂PO₄, such as a mixture of PEG having an average molecular weight in a range of from about 1000 to about 20,000, e.g. PEG6000 and KH₂PO₄, such as KH₂PO₄, in the molar concentration from 0.1 M to 10 M, such as from 0.1 M to 2 M, e.g. 0.1 M to 0.5 M, 0.33 M to 1 M, or even at 0.1 M, 0.33 M, 0.5 M or 1 M. Moreover, the release enhancing agent may be a mixture of polymers, such as PEG and poloxamer, e.g. PEG6000 and poloxamer 188.

In a further embodiment the release enhancing agent may be a mixture of a poloxamer and an inorganic salt. In a further embodiment the release enhancing agent may be a mixture of a poloxamer and an inorganic aqueous hydrogen phosphate.

The composition of the present invention comprising a porous silicium dioxide and a release enhancing agent shows, when compressed into a tablet, good and consistent release. A composition comprising a porous silicium dioxide, which has not been treated with a release enhancing agent increase its disintegration rate over time, whereas fast disintegration of the tablet is maintained for regardless of the type of treatment with a release enhancing agent. To optimize disintegration the composition of the present invention comprising a porous silicium dioxide, a release enhancing agent and compressed into a tablet, is mixed with a disintegrant before making the tablet. Thus, in an embodiment the loadable solid porous composition of the present invention, such as the tablet, comprises a porous silicium dioxide, a release enhancing agent, and a disintegrant. Such a disintegrant may be selected from croscarmellose sodium, alginic acid or alginates, microcrystalline cellulose, hydroxypropyl cellulose and other cellulose derivatives, crospovidone, polacrillin potassium, sodium starch glycolate, starch, pregelatinized starch, and carboxymethyl starch, typically croscarmellose sodium.

Some experiments have shown that the disintegration time is independent of disintegrant concentration to as low as at least 2.5% w/w (% of the loadable tablet) in tablets wherein the porous silicium dioxide has been treated with a release enhancing agent. In particular, the disintegration time of KH₂PO₄ treated porous silicium dioxide is substantially maintained with a disintegrant concentration to as low as at least 2.5% w/w (% of the loadable tablet)

Accordingly, in an embodiment of the present invention the tablet comprises a disintegrant, such as any one of the above mentioned, in a concentration from 1% w/w to 20% w/w, such as from 2% w/w to 10% w/w, or 2.5% w/w to 5% w/w (based on the total weight of the composition before loading).

Besides having porous silicium dioxide, a release enhancing agent, and optionally a disintegrant in the composition, whether it is loaded or not, the composition may further comprise a non-toxic excipient or carrier. Typically, such a non-toxic excipient or carrier is selected from a pharmaceutically acceptable excipient or carrier. In another embodiment, such a non-toxic excipient or carrier is selected from a nutritionally acceptable excipient or carrier.

Thus, the loadable composition, e.g. tablet, granulate or capsule, may of course also contain other pharmaceutically or nutritionally acceptable excipients or carriers such as those normally employed in the manufacturing of compositions, e.g. tablets, granulates or capsules.

Examples of such excipients or carriers are fillers, diluents, binders, lubricants, glidants, enhancers, wetting agents, solubilizing agents, surfactants, antioxidants, and metal scavengers.

In the present context the terms: “pharmaceutically acceptable excipient or carrier” and “nutritionally acceptable excipient or carrier” are intended to denote any material, which is inert in the sense that it substantially does not have any therapeutic and/or prophylactic effect or nutritional effect per se. Such an excipient or carrier may be added with the purpose of making it possible to obtain a pharmaceutical or a nutritional composition, which has acceptable technical properties.

Examples of suitable excipients or carriers for use in a loadable composition, such as tablet, according to the invention include without limitation fillers, diluents, binders, lubricants or a mixture thereof. As the composition or solid dosage form according to the invention may be used for different purposes, the choice of excipient or carrier is normally made taken such different uses into considerations. Other pharmaceutically or nutritionally acceptable excipients for suitable use are e.g. acidifying agents, alkalizing agents, preservatives, antioxidants, buffering agents, chelating agents, coloring agents, complexing agents, emulsifying and/or solubilizing agents, flavors, humectants, sweetening agents, wetting agents etc. Examples of suitable fillers, diluents and/or binders include lactose (e.g. spray-dried lactose, α-lactose, β-lactose, Tabletose®, various grades of Pharmatose®, Microtose® or Fast-Floc®), microcrystalline cellulose (various grades of Avicel®, Elcema®, Vivacel®, Ming Tai® or Solka-Floc®), hydroxypropylcellulose, L-hydroxypropylcellulose (low substituted), hydroxypropyl methylcellulose (HPMC) (e.g. Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen etc.

Examples of metal scavengers are e.g. tartaric acid, citric acid, oxalic acid, EDTA and salts thereof, DPTA (Diethylenetriaminepentaacetic Acid) and salts thereof.

Examples of antioxidants are e.g. BHT, BHA, propyl gallate, tocopherols, TBHQ (t-butyl hydroquinone), ascorbyl palmitate.

Specific examples of diluents are e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of binders are e.g. acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch etc.

Glidants and lubricants may also be included in the tablet. Examples include stearic acid, magnesium stearate, calcium stearate or other metallic stearate, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients or carriers which may be included in a loadable composition, such as tablet, of the invention are e.g. flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

Other additives in a composition or a solid dosage form according to the invention may be antioxidants like e.g. ascorbic acid, ascorbyl palmitate, butylated hydroxyanisole, butylated hydroxytoluene, hypophosphorous acid, monothioglycerol, potassium metabisulfite, propyl gallate, sodium formaldehylde sulfoxylate, sodium metabisulfite, sodium thiosulfate, sulfur dioxide, tocopherol, tocopherol acetate, tocopherol hemisuccinate, TPGS or other tocopherol derivatives, etc. The carrier composition may also contain e.g. stabilising agents. The concentration of an antioxidant and/or a stabilizing agent in the carrier composition is normally from about 0.1% w/w to about 5% w/w.

In a further aspect the present invention relates to a loadable solid porous composition comprising a porous silicium dioxide and a release enhancing agent loaded with a pharmaceutically acceptable oily substance.

The above embodiments as well as the embodiments to be described hereunder should be seen as referring to any one of the aspects described herein, whether it is compositions, such as granulates, capsules or tablets, or methods of preparing the same, as well as any one of the embodiments described herein unless it is specified that an embodiment relates to a certain aspect or aspects of the present invention.

In a further embodiment the composition is being formulated in a capsule. Thus, whether the composition is a granulate or tablets such can be formulated and contained in a capsule.

The pharmaceutically acceptable oily substance is present in a concentration of about 5% w/w or more such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50% w/w or more, about 60% w/w or more or about 70% or more (based on the total weight of the composition after loading.

The pharmaceutically acceptable oily substance comprises an edible or inedible oil or fat. Typically, the pharmaceutically acceptable oily substance comprises an edible oil or fat having a polyunsaturated fatty acids content of at least 10% w/w, typically a triglyceride content of at least 10% w/w, such as a triglyceride content of about 20% w/w. The edible oil or fat may be selected from a vegetable oil or fat or an animal oil or fat, or is a chemically modified or synthetic derived oil or fat. In a certain embodiment the oily substance is selected from a SMEDDS or NSEDDS. In a particular embodiment the edible oil or fat is selected from apricot oil, almond oil, avocado oil, castor oil, coconut fat, cocoa butter, corn oil, cotton seed oil, grape seed oil, jojoba oil, linseed oil, maize oil, olive oil, palm oil, peanut oil, poppy seed oil, rape seed oil, sesame oil, soybean oil, sunflower oil, thistle seed oil, walnut oil, wheat germ oil, hydrogenated peanut oil, hydrogenated palm kernels oil, hydrogenated cottonseed oil, hydrogenated soya oil, hydrogenated castor oil, hydrogenated coconut oil, beef tallow, lard, tall oil, whale oil, fish oil, omega-3 fatty acids, eicosapentaenoic acid (EPA), docosahexaenoic acid (DHA), EPA ethyl ester, DHA ethyl ester and free fatty acids thereof and mixtures thereof, for example any mixture of EPA ethyl ester and DHA ethyl ester.

The pharmaceutically acceptable oily substance to be loaded into the composition of the present invention, such as tablet or granulate, will normally have a viscosity of at the most about 600 mPa sec at a temperature of at the most about 150° C., since his will ensure the most expedient flow of the immiscible oil into the composition, such as tablet, by the capillary forces. Another parameter of the pharmaceutically acceptable oily substances the melting point which may be form at least about minus (−) 30° C. and at the most about 100° C., such as a melting point of about minus (−) 25° C. to about 70° C.

In order to provide a reliable pharmaceutical or nutritional composition to be given to mammals, such has human subjects, the pharmaceutically acceptable oily substance must be released from the loaded composition, and in particular must be released in a consistent and preferably high and consistent manner. Since the oily substance contained in the porous composition, e.g. tablet, and the media, such as human bodily fluids, are immiscible then the media has to replace the oily substance contained in the porous composition. This can happen by capillary pressure if the media has a better wettability of the porous surface than the oily substance. The capillary pressure arises in the small pores in the porous material. The driving force behind the replacement of the oily substance with the media is the difference in interfacial pressure. Thus, the pharmaceutically acceptable oily substance is released from the composition upon contact with an aqueous environment in an amount of at least 60% w/w, such as at least 70% w/w, at least 75% w/w, at least 80% w/w, at least 85% w/w, at least 90% w/w, at least 95% w/w, at least 99% w/w after storage for one week.

The loadable solid porous composition comprising a porous silicium dioxide and a release enhancing agent, and optionally a disintegrant, is typically loaded with the pharmaceutically acceptable oily substance as the only active ingredient all though a mixture of such oily substances is contemplated. Besides the pharmaceutically acceptable oily substance to be loaded into the composition, the composition may further be loaded with a liquid non-toxic excipient or carrier. Typically, such a liquid non-toxic excipient or carrier is selected from a pharmaceutically or nutritionally acceptable liquid excipient or carrier. In an embodiment the composition is selected from a pharmaceutically acceptable composition. In another embodiment the composition is selected from a nutritionally acceptable composition.

Since the loaded composition, such as tablet, granulate or capsule, is intended to oral ingestion by a mammal, such as human subject, the composition of the present invention should preferably be weighing from 100 mg to 5000 mg, such as from 200 mg to 1000 mg, or from 60 mg to 1500 mg in dried form.

The most efficient way of securing a uniform distribution of the release enhancing agent in a tablet is to first make a granulate of the porous silicium dioxide and the release enhancing agent and then compact, such as compress or mold, the obtained granulate to a tablet. However, it is also possible to simply make a tablet comprising compressed or molded porous silicium dioxide and then by applying force press the release enhancing agent into the tablet until such agent is uniformly distributed in the tablet. Accordingly, in a further aspect the present invention relates to a loadable solid porous tablet comprising a porous silicium dioxide wherein a release enhancing agent is compressed and uniformly distributed into said tablet.

Hereafter the tablet is ready to be loaded with the pharmaceutically acceptable oily substance.

Thus, in a still further aspect the present invention relates to a solid porous tablet comprising a porous silicium dioxide wherein a release enhancing agent is compressed and uniformly distributed into said tablet and the tablet is loaded with a pharmaceutically acceptable oily substance.

As it is the treatment of the porous silicium dioxide with the release enhancing agent that provides the efficient release of oily substance from the final composition, the intermediate composition, that is the granulate is also a part of the present invention.

Accordingly, a further aspect of the present invention provides a solid porous granulate comprising a porous silicium dioxide and a release enhancing agent.

As explained herein, the granulate may be compressed into tablets and then be loaded with the pharmaceutically acceptable oily substance, however, the granulate comprising a porous silicium dioxide and a release enhancing agent may also be used to absorb the pharmaceutically acceptable oily substance, whereafter the obtained granulate is either loaded into a capsule or is compressed into a tablet.

Moreover, a further aspect of the present invention provides a solid porous granulate comprising a porous silicium dioxide, a release enhancing agent and a pharmaceutically acceptable oily substance.

As already explained herein, the treatment of the porous silicium dioxide with the release enhancing agent is important in order to provide the final composition, such as a tablet or granulate, which release the pharmaceutically acceptable oily substance in a high and consistent manner over time.

Thus, in a further aspect the present invention concerns a method for the preparation of a loadable solid porous granulate comprising a porous silicium dioxide and a release enhancing agent, comprising the steps of:

i) preparing a granulate of the porous silicium dioxide and the release enhancing agent,
ii) optionally, heating the granules for a sufficient time at a suitable temperature,
iii) providing the loadable solid porous granulate.

In an embodiment of the method for the preparation of a loadable solid porous granulate comprising a porous silicium dioxide and a release enhancing agent step i) is carried out by spraying the release enhancing agent onto granules of porous silicium dioxide. In another embodiment of step i) the porous silicium dioxide and the release enhancing agent are mixed for a sufficient time, such as 5 min to 1 hour, typically 3 to 10 minutes to provide the granulate. Shorter time is possible depending on the mixing gear or equipment.

When the release enhancing agent is an inorganic salt, e.g. NaCl, or an inorganic aqueous hydrogen phosphate, e.g. KH₂PO₄, it is suitable in step i) to dissolve the inorganic salt or the inorganic aqueous hydrogen phosphate in water and mix it with the granulate of porous silicium dioxide, and then evaporate the water.

In the situation where the release enhancing agent is a mixture of an inorganic salt, e.g. NaCl, or an inorganic aqueous hydrogen phosphate, e.g. KH₂PO₄, and a polymer, such as PEG or poloxamer, then it is suitable in step i) to first mix the porous silicium dioxide with the inorganic salt or the inorganic aqueous hydrogen phosphate and then treat the obtained mixture with the polymer, preferably in a fluid bed.

The granules thus provided may be used as is or may be further processed by heating. Thus, in a further embodiment of the method for the preparation of a loadable solid porous granulate comprising a porous silicium dioxide and a release enhancing agent no heating is performed. In another embodiment step ii) is carried out by heating the granules for a sufficient time at a suitable temperature. The heating may be performed at temperatures from room temperature up to the temperature where the release enhancing agent starts to evaporate from the granulate, but is typically from 50 to 150° C., such as 80° C. to 110° C. The time of heating may vary since the granulate should be dry before further processing, but is typically from 30 min to 24 hours, such as 3 to 20 hours. After heating the granulate is typically cooled. The present invention also concerns a loadable solid porous granulate comprising a porous silicium dioxide and a release enhancing agent, obtainable by the method as described above.

A further aspect of the present invention concerns a method for the preparation of a solid porous granulate comprising a porous silicium dioxide, a release enhancing agent and a pharmaceutically acceptable oily substance, comprising the steps of:

i) preparing a granulate of the porous silicium dioxide and the release enhancing agent,
ii) optionally, heating the granules for a sufficient time at a suitable temperature,
iii) loading the pharmaceutically acceptable oily substance into the granules, optionally until the granules are saturated,
iv) optionally, heating the granules for a sufficient time at a suitable temperature,
v) providing the solid porous granulate.

In an embodiment of the method for the preparation of a loadable solid porous granulate comprising a porous silicium dioxide, a release enhancing agent and a pharmaceutically acceptable oily substance, step i) is carried out by spraying the release enhancing agent onto granules of porous silicium dioxide. In another embodiment of step i) the porous silicium dioxide and the release enhancing agent are mixed for a sufficient time, such as 3 to 10 min, or 5 min to 1 hour to provide the granulate.

When the release enhancing agent is an inorganic salt, e.g. NaCl, or an inorganic aqueous hydrogen phosphate, e.g. KH₂PO₄, it is suitable in step i) to dissolve the inorganic salt or the inorganic aqueous hydrogen phosphate in water and mix it with the granulate of porous silicium dioxide, and then evaporate the water.

In the situation where the release enhancing agent is a mixture of an inorganic salt, e.g. NaCl, or an inorganic aqueous hydrogen phosphate, e.g. KH₂PO₄, and a polymer, such as PEG or poloxamer, then it is suitable in step i) to first mix the porous silicium dioxide with the inorganic salt or the inorganic aqueous hydrogen phosphate and then treat the obtained mixture with the polymer, preferably in a fluid bed.

The granules thus provided may be used as is or may be further processed by heating. Thus, in a further embodiment of the method for the preparation of a loadable solid porous granulate comprising a porous silicium dioxide, a release enhancing agent and a pharmaceutically acceptable oily substance no heating is performed in step ii). In another embodiment step ii) is carried out by heating the granules for a sufficient time at a suitable temperature. The heating may be performed at temperatures from room temperature up to the temperature where the release enhancing agent starts to evaporate from granulate, but is typically from 50 to 150° C., such as 80° C. to 110° C. The time of heating may vary since the granulate should be dry before further processing, but is typically from 30 min to 24 hours, such as 3 to 20 hours. After heating, granulate is typically cooled. In step iii) the pharmaceutically acceptable oily substance is loaded into granulate, and this may be done by providing a surplus of the oily substance and waiting until granulate is saturated with the pharmaceutically acceptable oily substance. Typically in step iii) the pharmaceutically acceptable oily substance is loaded to about 95% of the loading capacity, such as about 90%, about 80%, about 70%, preferably the oily substance is loaded from 80% to 100% of the loading capacity. The granules loaded with the pharmaceutically acceptable oily substance may be used as is or may be further processed by heating. Thus, in a further embodiment of the method for the preparation of a loadable solid porous granulate comprising a porous silicium dioxide, a release enhancing agent and a pharmaceutically acceptable oily substance no heating is performed in step iv). In another embodiment step iv) is carried out by heating the granules for a sufficient time at a suitable temperature. The heating may be performed at temperatures from room temperature up to the boiling point of the pharmaceutically acceptable oily substance, but is typically from 50 to 150° C., such as 80° C. to 110° C. The time of heating may vary since the granulate should be dry before further processing, but is typically from 30 min to 24 hours, such as 3 to 20 hours. After heating the granulate is typically cooled. The present invention also concerns a solid porous granulate comprising a porous silicium dioxide, a release enhancing agent and a pharmaceutically acceptable oily substance, obtainable by the method as described above.

In a further aspect, the present invention relates to a method for the preparation of a capsule comprising a solid porous granulate comprising a porous silicium dioxide, a release enhancing agent and a pharmaceutically acceptable oily substance, the method comprising the steps of:

i) preparing a granulate of the porous silicium dioxide and the release enhancing agent,
ii) optionally, heating the granules for a sufficient time at a suitable temperature,
iii) loading the pharmaceutically acceptable oily substance into the granules, optionally until the granules are saturated,
iv) optionally, heating the granules for a sufficient time at a suitable temperature,
v) providing the solid porous granulate, and
vi) filling the solid porous granulate into the capsules.

In an embodiment of the method for the preparation of the capsule, step i) is carried out by spraying the release enhancing agent onto granules of porous silicium dioxide. In another embodiment of step i) the porous silicium dioxide and the release enhancing agent are mixed for a sufficient time, such as 3 min to 1 hour, e.g. 3 to 10 min to provide the granulate.

When the release enhancing agent is an inorganic salt, e.g. NaCl, or an inorganic aqueous hydrogen phosphate, e.g. KH₂PO₄, it is suitable in step i) to dissolve the inorganic salt or the inorganic aqueous hydrogen phosphate in water and mix it with the granulate of porous silicium dioxide, and then evaporate the water.

In the situation where the release enhancing agent is a mixture of an inorganic salt, e.g. NaCl, or an inorganic aqueous hydrogen phosphate, e.g. KH₂PO₄, and a polymer, such as PEG or poloxamer, then it is suitable in step i) to first mix the porous silicium dioxide with the inorganic salt or the inorganic aqueous hydrogen phosphate and then treat the obtained mixture with the polymer, preferably in a fluid bed.

The granules thus provided may be used as is or may be further processed by heating. Thus, in a further embodiment of the method for the preparation of the capsule no heating is performed in step ii). In another embodiment step ii) is carried out by heating the granules for a sufficient time at a suitable temperature. The heating may be performed at temperatures from room temperature up to the temperature where the release enhancing agent starts to evaporate from the granules, but is typically from 50 to 150° C., such as 80° C. to 110° C. The time of heating may vary since the granules (granulate) should be dry before further processing, but is typically from 30 min to 24 hours, such as 3 to 20 hours. After heating the granules (granulate) are typically cooled. In step iii) the pharmaceutically acceptable oily substance is loaded into the granules (granulate), and this may be done by providing a surplus of the oily substance and waiting until the granules (granulate) are saturated with the pharmaceutically acceptable oily substance. Typically, in step iii) the pharmaceutically acceptable oily substance is loaded to about 95% of the loading capacity, such as about 90%, about 80%, about 70%, preferably the oily substance is loaded from 80% to 100% of the loading capacity. The granules loaded with the pharmaceutically acceptable oily substance may be used as is or may be further processed by heating. Thus, in a further embodiment of the method for the preparation of the capsule no heating is performed in step iv). In another embodiment step iv) is carried out by heating the granules for a sufficient time at a suitable temperature. The heating may be performed at temperatures from room temperature up to the boiling point of the pharmaceutically acceptable oily substance, but is typically from 50 to 150° C., such as 80° C. to 110° C. The time of heating may vary since the granulate should be dry before further processing, but is typically from 30 min to 24 hours, such as 3 to 20 hours. After heating, the granules (granulate) are typically cooled. After the optional heating, the solid porous granulate comprising a porous silicium dioxide, a release enhancing agent and a pharmaceutically acceptable oily substance, is optionally sieved to provide a desired size of the granulate particles, then the granules (granulate) are filled into capsules in step vi), such as gelatin capsules. The present invention also concerns a capsule comprising a solid porous granulate comprising a porous silicium dioxide, a release enhancing agent and a pharmaceutically acceptable oily substance, obtainable by the method as described above.

In a further aspect, the present invention relates to a method for the preparation of a loadable solid porous tablet comprising a porous silicium dioxide and a release enhancing agent, and optionally a disintegrant, the method comprising the steps of:

i) preparing a granulate of the porous silicium dioxide and the release enhancing agent, and optionally a disintegrant,
ii) optionally, heating the granules for a sufficient time at a suitable temperature,
iii) providing the loadable solid porous granulate,
iv) compressing the granulate into loadable tablets,

or alternatively

a) preparing a granulate of the porous silicium dioxide, and optionally a disintegrant,
b) optionally, heating the granules for a sufficient time at a suitable temperature,
c) compressing the granulate into loadable tablets,
d) optionally, heating the tablets for a sufficient time at a suitable temperature,
e) uniformly distributing the release enhancing agent into the tablet by force, such as compression, e.g. compression under pressure,
f) providing the loadable tablets.

In an embodiment of the method for the preparation of the loadable tablet, no disintegrant is provided in step i). In another embodiment of the method for the preparation of the loadable tablet a disintegrant is provided in step i). In a further embodiment of the method for the preparation of the loadable tablet, step i) is carried out by spraying the release enhancing agent onto granules of porous silicium dioxide, and optionally the disintegrant. In another embodiment of step i) the porous silicium dioxide and the release enhancing agent are mixed for a sufficient time, such as 3 min to 1 hour, e.g. 3 to 10 min to provide the granulate, optionally comprising a disintegrant.

When the release enhancing agent is an inorganic salt, e.g. NaCl, or an inorganic aqueous hydrogen phosphate, e.g. KH₂PO₄, it is suitable in step i) to dissolve the inorganic salt or the inorganic aqueous hydrogen phosphate in water and mix it with the granulate of porous silicium dioxide, and optionally the disintegrant, and then evaporate the water.

Alternatively, in step e) when the release enhancing agent is an inorganic salt, e.g. NaCl, or an inorganic aqueous hydrogen phosphate, e.g. KH₂PO₄, it is suitable to dissolve the inorganic salt or the inorganic aqueous hydrogen phosphate in water before using force.

In the situation where the release enhancing agent is a mixture of an inorganic salt, e.g. NaCl, or an inorganic aqueous hydrogen phosphate, e.g. KH₂PO₄, and a polymer, such as PEG or poloxamer, then it is suitable in step i) to first mix the porous silicium dioxide with the inorganic salt or the inorganic aqueous hydrogen phosphate and then treat the obtained mixture with the polymer, preferably in a fluid bed.

Alternatively, in step e) when the release enhancing agent is a mixture of an inorganic salt, e.g. NaCl, or an inorganic aqueous hydrogen phosphate, e.g. KH₂PO₄, and a polymer, such as PEG or poloxamer, then it is suitable to first apply the inorganic salt or the inorganic aqueous hydrogen phosphate by force and then treat the obtained tablet with the polymer by force.

Alternatively, the steps of providing the loadable tablet of the present invention may be performed in a different order. In an embodiment of the method for the preparation of the loadable tablet, no disintegrant is provided in step a). In another embodiment of the method for the preparation of the loadable tablet a disintegrant is provided in step a).

The granules thus provided, comprising porous silicium dioxide and optionally a disintegrant (but no release enhancing agent) may be used as is or may be further processed by heating. Thus, in a further embodiment of the method for the preparation of the loadable tablet no heating is performed in step b). In another embodiment step b) is carried out by heating the granules for a sufficient time at a suitable temperature. The heating may be performed at temperatures from room temperature up to the temperature where porous silicium dioxide starts to degrade, but is typically from 50 to 150° C., such as 80° C. to 110° C. The time of heating may vary since the granulate should be dry before further processing, but is typically from 30 min to 24 hours, such as 3 to 20 hours. After heating the granulate is typically cooled. After the optional heating, the solid porous granulate comprising a porous silicium dioxide, and optionally a disintegrant, is optionally sieved to provide a desired size of the granulate particles, and then in step c) the granulate is compressed into the loadable tablets. In a further embodiment of the method for the preparation of the loadable tablet no heating is performed in step d). In another embodiment step d) is carried out by heating the tablet for a sufficient time at a suitable temperature. The heating may be performed at temperatures from room temperature up to the temperature where porous silicium dioxide starts to degrade, but is typically from 50 to 150° C., such as 80° C. to 110° C. The time of heating may vary since the tablet should be dry before further processing, but is typically from 30 min to 24 hours, such as 3 to 20 hours. After heating, the tablet is typically cooled. After, the optional heating in step d), the release enhancing agent in step e) is uniformly distributed in the tablet by force, such as compression, e.g. compression under pressure. Hereafter, the tablets are now ready for loading of the pharmaceutically acceptable oily substance.

Experiments performed have shown that in order to obtain a high and consistent release of oily substance from the final tablet, it is better to perform the heating in step ii), b), and/or d). In particular, performing the heating of the tablet provides better results than only heating the granulate. Thus, in a further embodiment, heating is performed in step ii). In a further embodiment heating is performed in step b). In a still further embodiment heating is performed in step d). It is noted that heating may be carried out in both step b) and d), or just step b) or d). The present invention also concerns a loadable solid porous tablet comprising a porous silicium dioxide and a release enhancing agent, and optionally a disintegrant, obtainable by the method as described above.

The preparation of the loadable tablet may be carried out as described above and the tablet may then be loaded with a pharmaceutically acceptable oily substance as described. The pharmaceutically acceptable oily substance is loaded to about 95 of the loading capacity, such as about 90%, about 80%, about 70%, preferably the oily substance is loaded from about 80% to 100% of the loading capacity.

In a further aspect the present invention concerns a method for the preparation of a solid porous tablet comprising a porous silicium dioxide, a release enhancing agent, optionally a disintegrant, and a pharmaceutically acceptable oily substance. As described several ways of obtaining the loadable tablets exists, and are without limitation all relevant for the further processing to provide the loaded tablet.

The method of preparing the loaded tablet, comprises the steps of:

i) preparing a loadable tablet,
ii) optionally, heating the tablet for a sufficient time at a suitable temperature,
iii) loading the pharmaceutically acceptable oily substance into the tablet, optionally until the tablet is saturated,
iv) optionally heating the tablet for a sufficient time at a suitable temperature, to provide the loaded solid porous tablet.

The tablets thus provided in step i) may be used as is or may be further processed by heating. Thus, in a further embodiment of the method for the preparation of the loaded tablet no heating is performed in step ii). In another embodiment step ii) is carried out by heating. The heating may be performed at temperatures from room temperature up to the temperature where porous silicium dioxide starts to degrade, but is typically from 50 to 150° C., such as 80° C. to 110° C. The time of heating may vary since the tablet should be dry before further processing, but is typically from 30 min to 24 hours, such as 3 to 20 hours. After heating the tablet is typically cooled. In step iii) the pharmaceutically acceptable oily substance is loaded into the tablet, such as by placing the loadable tablet in a container containing the pharmaceutically acceptable oily substance. In order to provide a loaded tablet which is saturated with the pharmaceutically acceptable oily substance, this is typically done by providing a surplus of the oily substance and waiting until the tablet has absorbed the pharmaceutically acceptable oily substance. Typically, the pharmaceutically acceptable oily substance is loaded to about 95% of the loading capacity, such as about 90%, about 80%, about 70%, preferably the oily substance is loaded from about 80% of the loading capacity to 100%. The tablets thus provided in step iii) may be used as is or may be further processed by heating. Thus, in a further embodiment of the method for the preparation of the loaded tablet no heating is performed in step iv). In another embodiment step iv) is carried out by heating. The heating may be performed at temperatures from room temperature up to the boiling point of the pharmaceutically acceptable oily substance, but is typically from 50 to 150° C., such as 80° C. to 110° C. The time of heating may vary since the tablet should be dry before use, but is typically from 30 min to 24 hours, such as 3 to 20 hours. After heating, the tablet is typically cooled.

Experiments performed have shown that in order to obtain a high and consistent release of oily substance from the final tablet, it is better to perform the heating in step ii). In particular, performing the heating of the loadable tablet provides better high and consistent release of the pharmaceutically acceptable oily substance from the final tablet. Moreover, by treating the porous silicium oxide with a release enhancing agent, such as PEG and then heating the provided tablet at a temperature from 50 to 150° C., such as 80° C. to 150° C., or 50 to 110° C., results in a high stability of the pharmaceutically acceptable oily substance when loaded into the tablet. Accordingly, in a further embodiment, heating is performed in step ii). It is noted that heating may be carried out in both step ii and iv). After heating the tablet is typically cooled.

The heating of the tablet of the present invention which has been treated with polymer, such as PEG6000, prevents interactions between the pharmaceutically acceptable oily substance and the porous silicium oxide, such as Neusilin. This leads to high and consistent release of oily substance and prevents chemical degradation of the oily substance.

In a further embodiment of the method for the preparation of a solid porous tablet comprising a porous silicium dioxide, a release enhancing agent, optionally a disintegrant, and a pharmaceutically acceptable oily substance, the loading in step iii) is performed by placing the tablet in an excess amounts of the pharmaceutically acceptable oily substance for a sufficient amount of time. Typically, the pharmaceutically acceptable oily substance is loaded to about 95% of the loading capacity, such as about 90%, about 80%, about 70%, preferably the oily substance is loaded from about 80% of the loading capacity to 100%. Such loading of oily substance maybe performed under pressure. When loading is performed under pressure the time of loading may be considerably reduced. If loading is performed in a surplus of pharmaceutically acceptable oily substance then, typically, the time period of loading the pharmaceutically acceptable oily substance is from 30 min to 3 hours, preferably from 30 min to 1 hour. The present invention also concerns a solid porous tablet comprising a porous silicium dioxide, a release enhancing agent, optionally a disintegrant, and a pharmaceutically acceptable oily substance, obtainable by the method as described above.

In a further aspect the present invention provides use of a release enhancing agent for treating a porous silicium dioxide to provide the porous silicium dioxide which has an increased and maintained ability to release a pharmaceutically acceptable oily substance from the porous silicium dioxide.

### LIST OF EMBODIMENTS

1. A loadable solid porous composition comprising a porous silicium dioxide (silicon dioxide) and a release enhancing agent.
1A. A loadable solid porous composition consisting essentially of a porous silicium dioxide pre-deposited with a release enhancing agent.
2. The composition of embodiment 1, wherein the composition is a compacted, such as a compressed or molded tablet that has a hardness of 20 N or more, typically, the tablet has a hardness of 25 N or more, about 30 N or more, about 35 N or more, about 40 N or more, about 45 N or more, about 50 N or more, about 60 N or more, about 70 N or more, about 90 N or more, about 100 N or more, about 150 N or more or about 200 N, typically from about 30 N to about 150 N, such as 30 N to 100 N.
3. The composition of embodiment 1 comprising a granulate.
4. The composition of any one of embodiments 1-3, wherein said composition has a porosity of 30% v/v or more, such as 40% v/v or more, 50% v/v or more, 60% v/v or more, 70% v/v or more, 80% v/v or more, or 90% v/v or more.
5. The composition of any one of the preceding embodiments wherein the porous silicium dioxide is present in a concentration of about 20% w/w or more, about 25% w/w or more, about 30% w/w or more, about 35% w/w or more, about 40% w/w or more, about 45% w/w or more, about 50 w/w or more, about 60% w/w or more, about 70% or more, about 80% or more, about 90% or more, about 95% or more, or about 98% or more in the unloaded composition.
6. The composition of any one of the preceding embodiments, wherein the porous silicium dioxide is selected from magnesium aluminum metasilicate, magnesium aluminum silicate, aluminum metasilicate, Neusilin SG2, Neusilin US2, and mixtures thereof, such as magnesium aluminum metasilicate.
7. The composition of any one of the preceding embodiments, wherein the release enhancing agent is present in a concentration of about 2% w/w or more, such as about 5% w/w or more, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 60% w/w or more or about 70% or more (based on the total weight of the composition before loading), such as from 5% to 70% w/w, e.g. from 35% to 50% w/w.
8. The composition of any one of the preceding embodiments wherein the release enhancing agent is selected from a polymer, an inorganic salt or an inorganic aqueous hydrogen phosphate and mixtures thereof.
9. The composition of embodiment 8 wherein the release enhancing agent is a polymer selected from a polyethylene glycol, a poloxamer, a polyethylene oxide, an alkyl cellulose, e.g. HPMC, or polyvinyl alcohol (PVA), polyvinyl acetate phthalate, polyvinyl acetate or mixtures thereof.
10. The composition of embodiment 9 wherein the polymer is a polyethylene glycol having an average molecular weight in a range of from about 400 to about 100,000, such as from about 400 to about 35,000 such as, e.g., from about 800 to about 35,000, from about 1,000 to about 35,000 such as, e.g., polyethylene glycol 1,000, polyethylene glycol 2,000, polyethylene glycol 3,000, polyethylene glycol 4,000, polyethylene glycol 5,000, polyethylene glycol 6000, polyethylene glycol 7,000, polyethylene glycol 8,000, polyethylene glycol 9,000 polyethylene glycol 10,000, polyethylene glycol 15,000, polyethylene glycol 20,000, or polyethylene glycol 35,000; and mixtures thereof; or the polymer is a poloxamer, such as Poloxamer 188, Poloxamer 237, Poloxamer 338 or Poloxamer 407 or other block copolymers of ethylene oxide and propylene oxide, and mixtures thereof, having an average molecular weight in a range of from about 400 to about 100,000, such as from about 400 to about 35,000 such as, e.g., from about 800 to about 35,000, from about 1,000 to about 35,000; and mixtures thereof.
11. The composition of embodiment 8 wherein the release enhancing agent is an inorganic salt, such as an alkaline salt, e.g. NaCl or is an inorganic aqueous hydrogen phosphate selected from alkali, such as sodium, potassium, magnesium hydrogen phosphate, or sodium, potassium, magnesium dihydrogen phosphate, phosphoric acid or phosphorous acid.
12. The composition of embodiment 11 wherein the release enhancing agent is potassium dihydrogen phosphate (KH₂PO₄) in a concentration from 0.1 M to 10 M, such as from 0.1 M to 2 M.
13. The composition of any one of the preceding claims wherein the release enhancing agent is selected from a mixture of a polymer and an inorganic salt, such as an alkaline salt, e.g. NaCl or an inorganic aqueous hydrogen phosphate, from a mixture of polymers, or from a mixture of salts.
14. The composition of any one of the preceding embodiments further comprising a disintegrant.
15. The composition of embodiment 14 wherein the disintegrant is selected from croscarmellose sodium, alginic acid or alginates, microcrystalline cellulose, hydroxypropyl cellulose and other cellulose derivatives, crospovidone, polacrillin potassium, sodium starch glycolate, starch, pregelatinized starch, and carboxymethyl starch.
16. The composition of any one of embodiments 14-15 wherein the concentration of disintegrant is from 1% w/w to 20% w/w such as 1% w/w to 10% w/w, such as 2% w/w to 15% w/w, such as 2.5% w/w to 8% w/w (based on the total weight of the composition before loading)
17. The composition of any one of the preceding embodiments, wherein the composition is in the form of a compressed tablet, and the release enhancing agent is uniformly distributed in the tablet.
18. The composition of any one of the preceding claims comprising a non-toxic excipient or carrier is selected from a pharmaceutically or nutritionally acceptable excipient or carrier.
19. The composition of any one of the preceding embodiments, wherein the composition is selected from a pharmaceutically or nutritionally acceptable composition.
20. The composition of any one of the preceding embodiments wherein the porous silicium dioxide and the release enhancing agent have been heated to a temperature from 50 to 150° C., such as 80° C. to 110° C., for 5 minutes to 24 hours.
21. The composition of embodiment 20 wherein the sufficient time is from 5 min to 24 hours, such as 10 min 20 hours, 15 min to 10 hours, or 30 min to 10 hours.
21A. A solid pharmaceutical composition comprising: (a) a porous silicium dioxide pre-deposited with a release enhancing agent; and (b) a pharmaceutically active substance loaded in the porous silicium dioxide, optionally dissolved or dispersed in a pharmaceutically acceptable oily substance.
21B. The composition of embodiment 21A, wherein the porous silicium dioxide has a particle size between 44 micron and 177 micron prior to pre-deposition with the release enhancing agent.
22. The composition of any one of the preceding embodiments loaded with a pharmaceutically acceptable oily substance optionally comprising a pharmaceutically active substance.
23. The composition of any one of the preceding embodiments being formulated in a capsule.
24. The composition of embodiment 21A, 21B, 22 or 23, wherein the pharmaceutically acceptable oily substance or the pharmaceutically active substance is present in a concentration of about 5% w/w or more such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50% w/w or more, about 60% w/w or more or about 70% or more (based on the total weight of the composition after loading).
25. The composition of any one of embodiments 21A-24, wherein the pharmaceutically acceptable oily substance or the pharmaceutically active substance comprises one or more structured triglycerides, mono-glycerides, fatty acids, or esters of fatty acids, or mixtures thereof.
26. The composition of any one of embodiments 21A-25, wherein the pharmaceutically acceptable oily substance has a polyunsaturated fatty acids content of at least 10% w/w, typically a triglyceride content of about 20% w/w.
27. The composition of any one of embodiments 21A-26, wherein the pharmaceutically acceptable oily substance or the pharmaceutically active substance is selected from a vegetable oil or fat, or an animal oil or fat, or is a chemically modified or synthetic derived oil or fat, or oil derivatives, or emulsions or SMEDDS or NSEDDS.
28. The composition of any one of embodiments 21A-27, wherein the pharmaceutically acceptable oily substance or the pharmaceutically active substance is selected from apricot oil, almond oil, avocado oil, castor oil, coconut fat, cocoa butter, corn oil, cotton seed oil, grape seed oil, jojoba oil, linseed oil, maize oil, olive oil, palm oil, peanut oil, poppy seed oil, rape seed oil, sesame oil, soybeen oil, sunflower oil, thistle seed oil, walnut oil, wheat germ oil, hydrogenated peanut oil, hydrogenated palm kernels oil, hydrogenated cottonseed oil, hydrogenated soya oil, hydrogenated castor oil, hydrogenated coconut oil, beef tallow, lard, tall oil, whale oil, fish oil, EPA ethyl ester, EPA, DHA, DHA ethyl ester and free fatty acids thereof, cod liver oil, flaxseed oil, palm kernel oil, safflower oil, shea nut oil and mixtures thereof, as well as structured triglycerides, mono-glycerides, fatty acids, or esters of fatty acids from said oils, or mixtures thereof.
29. The composition of any one of embodiments 21A-28, wherein the pharmaceutically acceptable oily substance has a melting point of at least about minus (−) 30° C. and at the most about 100° C., such as a melting point of about minus (−) 25° C. to about 70° C.
30. The composition of any one of embodiments 21A-29, wherein the pharmaceutically acceptable oily substance is released from the composition upon contact with an aqueous environment in an amount of at least 60% w/w, such as at least 70% w/w, at least 75% w/w, at least 80% w/w, at least 85% w/w, at least 90% w/w, at least 95% w/w, at least 99% w/w after storage for one week.
31. The composition of any one of embodiments 21A-30 further loaded with a liquid non-toxic excipient or carrier.
32. The composition of embodiment 31, wherein the liquid non-toxic excipient or carrier is selected from a pharmaceutically or nutritionally acceptable liquid excipient or carrier.
33. The composition of any one of embodiments 21A-32, wherein the composition is selected from a pharmaceutically or nutritionally acceptable composition.
34. The composition of any one of embodiments 21A-33 weighing from 100 mg to 5000 mg, such as from 200 mg to 1000 mg, or from 60 mg to 1500 mg in dried form.
35. A solid porous tablet comprising a porous silicium dioxide wherein a release enhancing agent is compressed and uniformly distributed into said tablet.
36. The solid porous tablet of embodiment 35 loaded with a pharmaceutically acceptable oily substance.
36A. The composition of any one of embodiments 21A-30, wherein the composition is in the form of a compressed tablet, and the release enhancing agent is uniformly distributed in the tablet.
36B. The composition of any one of embodiments 21A-30 and 36A, wherein the composition is prepared by(a) obtaining a granulate consisting essentially of the porous silicium dioxide and the release enhancing agent, and (b) loading the pharmaceutically active substance into the granules, until the pharmaceutically active substance is loaded to about 70% or more of the loading capacity.
37. A solid porous granulate capable of carrying a load consisting essentially of a porous silicium dioxide and a release enhancing agent.
38. A granulate comprising (a) a porous silicium dioxide pre-deposited with a release enhancing agent, and (b) a pharmaceutically active substance.
39. A method for the preparation of a loadable solid porous granulate comprising a porous silicium dioxide and a release enhancing agent, comprising the steps of:
i) preparing a granulate of the porous silicium dioxide and the release enhancing agent, such as by spraying the release enhancing agent onto granules of porous silicium dioxide,
ii) optionally, heating the granules at a temperature from 50 to 150° C., such as 80° C. to 110° C., from 30 min to 24 hours, such as 3 to 20 hours,
iii) providing the loadable solid porous granulate.
40. A method for the preparation of a solid porous granulate comprising a porous silicium dioxide, a release enhancing agent and a pharmaceutically acceptable oily substance, comprising the steps of:
i) preparing a granulate of the porous silicium dioxide and the release enhancing agent, such as by spraying the release enhancing agent onto granules of porous silicium dioxide,
ii) optionally, heating the granules at a temperature from 50 to 150° C., such as 80° C. to 110° C., from 30 min to 24 hours, such as 3 to 20 hours,
iii) loading the pharmaceutically acceptable oily substance into the granules, until the pharmaceutically acceptable oily substance is loaded to about 95% of the loading capacity, such as about 90%, about 80%, about 70%, preferably the oily substance is loaded from about 80% of the loading capacity to 100%,
iv) optionally, heating the granules at a temperature from 50 to 150° C., such as 80° C. to 110° C., from 30 min to 24 hours, such as 3 to 20 hours,
v) providing the solid porous granulate.
41. A method for the preparation of a capsule comprising a solid porous granulate comprising a porous silicium dioxide, a release enhancing agent and a pharmaceutically acceptable oily substance, the method comprising the steps of:
i) preparing a granulate of the porous silicium dioxide and the release enhancing agent, such as by spraying the release enhancing agent onto granules of porous silicium dioxide,
ii) optionally, heating the granules at a temperature from 50 to 150° C., such as 80° C. to 110° C., from 30 min to 24 hours, such as 3 to 20 hours,
iii) loading the pharmaceutically acceptable oily substance into the granules, until the pharmaceutically acceptable oily substance is loaded to about 95% of the loading capacity, such as about 90%, about 80%, about 70%, preferably the oily substance is loaded from about 80% of the loading capacity to 100%,
iv) optionally, heating the granules at a temperature from 50 to 150° C., such as 80° C. to 110° C., from 30 min to 24 hours, such as 3 to 20 hours,
v) providing the solid porous granulate, and
vi) filling the solid porous granulate into the capsules.
42. A method for the preparation of a loadable solid porous tablet comprising a porous silicium dioxide and a release enhancing agent, and optionally a disintegrant, the method comprising the steps of:
i) preparing a granulate of the porous silicium dioxide and the release enhancing agent, such as by spraying the release enhancing agent onto granules of porous silicium dioxide, and optionally a disintegrant,
ii) optionally, heating the granules at a temperature from 50 to 150° C., such as 80° C. to 110° C., from 30 min to 24 hours, such as 3 to 20 hours,
iii) providing the loadable solid porous granulate,
iv) compressing the granulate into loadable tablets,

or alternatively

a) preparing a granulate of the porous silicium dioxide, and optionally a disintegrant,
b) optionally, heating the granules at a temperature from 50 to 150° C., such as 80° C. to 110° C., from 30 min to 24 hours, such as 3 to 20 hours,
c) compressing the granulate into loadable tablets,
d) optionally, heating the tablets at a temperature from 50 to 150° C., such as 80° C. to 110° C., from 30 min to 24 hours, such as 3 to 20 hours,
e) uniformly distributing the release enhancing agent into the tablet by force, such as compression, e.g. compression under pressure,
f) providing the loadable tablets.
43. A method for the preparation of a solid porous tablet comprising a porous silicium dioxide, a release enhancing agent, optionally a disintegrant, and a pharmaceutically acceptable oily substance, the method comprising the steps of:
i) preparing a loadable tablet of embodiment 42,
ii) optionally, heating the tablet at a temperature from 50 to 150° C., such as 80° C. to 110° C., from 30 min to 24 hours, such as 3 to 20 hours,
iii) loading the pharmaceutically acceptable oily substance into the tablet until the pharmaceutically acceptable oily substance is loaded to about 95% of the loading capacity, such as about 90%, about 80%, about 70%, preferably the oily substance is loaded from about 80% of the loading capacity to 100%,
iv) optionally heating the tablet at a temperature from 50 to 150° C., such as 80° C. to 110° C., from 30 min to 24 hours, such as 3 to 20 hours, to provide the loaded solid porous tablet.
44. The method of embodiment 43 wherein the loading is performed by placing the tablet in an excess amounts of the pharmaceutically acceptable oily substance for a sufficient amount of time.
45. The method of embodiment 44 wherein the loading is performed under pressure, such as under reduced pressure.
46. The method of embodiments 44-45, wherein the time period of loading the pharmaceutically acceptable oily substance is from 30 min to 3 hours, preferably from 30 min to 1 hour.
47. A loadable solid porous granulate comprising a porous silicium dioxide and a release enhancing agent, obtainable by the method of embodiment 39.
48. A solid porous granulate comprising a porous silicium dioxide, a release enhancing agent and a pharmaceutically acceptable oily substance, obtainable by the method of embodiment 40.
49. A capsule comprising a solid porous granulate comprising a porous silicium dioxide, a release enhancing agent and a pharmaceutically acceptable oily substance, obtainable by the method of embodiment 41.
50. A loadable solid porous tablet comprising a porous silicium dioxide and a release enhancing agent, and optionally a disintegrant, obtainable by the method of embodiment 42.
51. A solid porous tablet comprising a porous silicium dioxide, a release enhancing agent, optionally a disintegrant, and a pharmaceutically acceptable oily substance, obtainable by the method of any one of embodiments 43-46.
52. Use of a release enhancing agent for treating a porous silicium dioxide to provide the porous silicium dioxide which has an increased and maintained ability to release a pharmaceutically acceptable oily substance from the porous silicium dioxide.
The invention is further illustrated in the following non-limiting examples.

### Materials

Magnesium aluminometasilicate (Al₂O₃.MgO.1.7SiO₂.xH₂O) sold under the trade name Neusilin® US2 by Fuji Chemical Industry Co.
Croscarmellose sodium, Ac-Di-Sol®

### Poloxamer 188, Pluronic® F68 (BASF)

Eicosapentaenoic acid (EPA)/docosahexaenoic acid (DHA) ethyl ester MCT oil (medium-chain fatty acid esters of glycerol sold under the trade name Viscoleo®

### REFERENCE COMPOSITIONS

### Reference Granulate and Tablet Compositions—Preparation and Loading with Oil Substance

A total of five inert loadable reference tablets (ref1, ref2, ref3, ref4, ref5) were prepared by mixing magnesium aluminometalsilicate with magnesium stearate and optionally croscarmellose sodium for 0.5 min in a Turbula mixer and compressing the blend into tablets using a single punch tablet press. The tablets were dried with paper and stored in a high density polyethylene (HDPE) bottle.

Each reference tablet was loaded with an oily substance using the method described in Materials and Methods.

| Composition | Ref1 | Ref2 | Ref3 | Ref4 | Ref5 |
|---|---|---|---|---|---|
| Mg aluminometasilicate (g) | 50.0 | 23.1 | 133.5 | 133.5 | 118.5 |
| Mg stearate (g) | 0.51 | 0.3 | 1.5 | 1.5 | 1.5 |
| Croscarmelllose sodium (g) | — | 6.61 | 15.0 | 15.0 | 30.0 |
| Total weight of inert tablets (g) | 50.5 | 30 | 150 | 150 | 150 |
| Corn oil loaded into tablets (g) | 101 | 52.3 | — | — | — |
| EPA/DHA ethyl ester loaded (g) | — | — | 300 | — | — |
| Viscoleo MCT oil loaded (g) | — | — | — | 197 | 186 |
| Total weight (g) | 151 | 82.3 | 450 | 347 | 336 |
| (oil loaded tablets) | | | | | |
| Tablet size (mm) | 12.5 | 12.5 | 12 | 9 | 9 |
| Weight of inert tablet (mg) | 274 | 275 | 250 | 137 | 137 |
| ‘Tablet hardness (N) | 40 | 40 | 60 | 50-60 | 50-60 |

### Example 1

### Loadable Tablets of the Invention Using Polyethylene Glycol (PEG 6000) as Release Enhancing Agent

Tablet compositions (inert tablets) of the invention according to Table 1 below were prepared as follows: Granules (granulate) were obtained by heating release enhancing agent PEG6000 to 75° C. and subsequent spraying onto magnesium aluminosilicate in a conventional fluidized bed for 11 minutes. The granules were mixed with magnesium stearate for 0.5 minutes in a Turbula mixer, and the blend was compressed into tablets using a single punch tablet press.

Compositions 1A and 1B: Granules were heated for 3 hours at 80° C. and then cooled prior to mixing with Mg sterate and tablet compression

Compositions 1C, 1D and 1E: Tablets were heated for 3 hours at 80° C. and then cooled.

Compositions 1B, 1C, 1D and 1E: The tablets were submerged into corn oil until fully loaded with oil. The tablets were then dried with paper and stored in a HDPE bottle.

The release test method was performed by gently stirring the tablets in 250 mL de-mineralized water by a magnet for 2 hours followed by drying to constant weight at 110° C. The weight difference between the oil loaded tablets and dried tablets after release relates to the oil released. The release test was carried out at day 1 and day 7 (1 week), respectively; (results are average; n=2).

The tablet compositions and the release results are shown in Table 1:

TABLE 1

| Composition | Ref1 | 1A | 1B | 1C | 1D | 1E |
|---|---|---|---|---|---|---|
| Mg alumino- | 50.0 | 30.0 | 30.0 | 30.0 | 40.0 | 45.0 |
| metasilicate (g) | | | | | | |
| Mg stearate (g) | 0.51 | 0.51 | 0.51 | 0.51 | 0.51 | 0.51 |
| PEG 6000 (g) | — | 20.0 | 20.0 | 20.0 | 10.0 | 5.0 |
| Total weight of | 50.51 | 50.51 | 50.51 | 50.51 | 50.51 | 50.51 |
| inert tablets (g) | | | | | | |
| Corn oil loaded | 101 | — | 48.6 | 59.3 | 78.0 | 89.9 |
| into tablets (g) | | | | | | |
| Total weight (g) | 151 | 50.51 | 99.1 | 110 | 129 | 140 |
| (oil loaded | | | | | | |
| tablets) | | | | | | |
| Tablet weight | 274 | 458 | 458 | 458 | 367 | 321 |
| (mg) | | | | | | |
| Release, Day 1 | 84% | 96 mg | 68% | 94% | 81% | 80% |
| Release, Day 7 | 34% | 78 mg | 57% | 82% | 51% | 34% |

Comp. 1A is an inert tablet (not loaded with oily substance). The release data shows that a minor amount of PEG6000 is released from the tablet. The release data for all other (oil loaded) compositions are adjusted for this simultaneous release of PEG.

### Example 2

### Loadable Tablets of the Invention Using Potassium Dihydrogenphosphate (KH₂PO₄) as Release Enhancing Agent

Tablet compositions (inert tablets) of the invention according to Table 2 were prepared as follows: A solution of KH₂PO₄ was gently mixed into magnesium aluminosilicate by spatula in a glass beaker. The granules were heated to constant weight at 110° C. for about 12 hours and then cooled prior to mixing with magnesium stearate for 0.5 minutes in a Turbula mixer. The blend was compressed into tablets using a single punch tablet press.

Compositions 2A, 2B, 2C, 2E and 2F: The tablets were submerged into corn oil until fully loaded with oil. The tablets were then dried with paper and stored in a HDPE bottle.

The release test method was performed as described in Example 1.

The tablet compositions and the release results are shown in Table 2:

TABLE 2

| Composition | Ref1 | 2A | 2B | 2C | 2D | 2E | 2F |
|---|---|---|---|---|---|---|---|
| Mg aluminometasilicate (g) | 50.0 | 48.7 | 44.1 | 39.4 | 44.1 | 44.1 | 47.5 |
| Mg stearate (g) | 0.51 | 0.51 | 0.51 | 0.51 | 0.51 | 0.51 | 0.51 |
| KH₂PO₄ (g) | — | 1.31 | 5.95 | 10.6 | 5.95 | 5.95 | 2.5 |
| Total weight of inert tabs (g) | 50.51 | 50.51 | 50.51 | 50.51 | 50.51 | 50.51 | 50.51 |
| Corn oil loaded (g) | 101 | 118 | 127 | 94.2 | — | 102 | 81.9 |
| Total weight (g) | 151 | 169 | 178 | 144.7 | 50.5 | 153 | 132 |
| (oil loaded tablets) | | | | | | | |
| Tablet weight (mg) | 274 | 275 | 275 | 275 | 275 | 275 | 275 |
| Tablet harness (N) | 40 | 40 | 40 | 40 | 40 | 40 | 40 |
| Release, Day 1 | 84% | 68% | 79% | 87% | 11 mg* | 87% | 78% |
| Release, Day 7 | 34% | 39% | 79% | 71% | n.a. | 74% | 67% |

Comp. 2D is an inert tablet (not loaded with oily substance). The release data shows that a minor amount of KH₂PO₄ is released from the tablet at day 1.

Comp. 2E: Loading is 150% of theoretical tablet loading capacity for even better distribution of the same amount of KH₂PO₄ in the silicate compared to comp. 2B.

Comp. 2F: The KH₂PO₄ was directly compressed into the tablet to investigate if substantial distribution of the salt in the tablet is required.

### Example 3

### Loadable Tablets of the Invention Using Polyethylene Glycol (PEG6000) and Potassium Dihydrogenphosphate (KH₂PO₄) as Release Enhancing Agents

Granules were prepared and heated as described in Example 2. PEG6000 was heated to 75° C. and subsequent sprayed onto the granules in a conventional fluidized bed for 11 minutes. The resulting granules were mixed with magnesium stearate in a Turbula mixer, and the blend was compressed into tablets using a single punch tablet press. The tablets were loaded with corn oil and tested in the release test as disclosed in Example 1 providing the results shown in Table 3.

TABLE 3

| Composition | Ref1 | 3A |
|---|---|---|
| Mg aluminometasilicate (g) | 50.0 | 27.0 |
| Mg stearate (g) | 0.51 | 0.51 |
| KH₂PO₄ (g) | — | 3.0 |
| PEG 6000 (g) | — | 20.0 |
| Total weight of inert tablets (g) | 50.51 | 50.51 |
| Corn oil loaded into tablets (g) | 101 | 52.7 |
| Total weight (g) | 151 | 103 |
| (oil loaded tablets) | | |
| Tablet weight (mg) | 274 | 458 |
| Release, Day 1 | 84% | **) |
| Release, Day 7 | 34% | **) |

**) Tablets disintegrated in about 1 hour.

### Example 4

### Loadable Tablets of the Invention Using a Poloxamer as Release Enhancing Agent Optionally in Combination with Other Enhancing Agents

Tablet compositions (inert tablets) 4A, 4B, 4C and 4D according to Table 4 were prepared as follows: Granules (granulate) were obtained by heating release enhancing agent poloxamer 188 to 75° C. and subsequent spraying onto magnesium aluminosilicate in a conventional fluidized bed for 11 minutes. The granules were mixed with magnesium stearate for 0.5 minutes in a Turbula mixer, and the blend was compressed into tablets using a single punch tablet press.

Comp. 4E was prepared in the same manner as described in Example 2 using phosphoric acid instead of KH₂PO₄.

Composition 4A: Granules were heated for 3 hours at 80° C. and then cooled prior to mixing with Mg sterate and tablet compression

Compositions 4B, 4C and 4D: Tablets were heated for 3 hours at 80° C. and then cooled.

The tablets were loaded with corn oil and tested in the release test described in Example 1 providing the results shown in Table 4.

TABLE 4

| Composition | Ref1 | 4A | 4B | 4C | 4D | 4E |
|---|---|---|---|---|---|---|
| Mg alumino- | 50.0 | 30.0 | 30.0 | 30.0 | 27.0 | 44.1 |
| metasilicate (g) | | | | | | |
| Mg stearate (g) | 0.51 | 0.51 | 0.51 | 0.51 | 0.51 | 0.51 |
| Poloxamer | — | 20.0 | 20.0 | 20.0 | 20.0 | — |
| 188 (g) | | | | | | |
| KH₂PO₄ (g) | — | — | — | — | 3.0 | — |
| H₃PO₄ (g) | — | — | — | — | — | 5.95 |
| Total weight of | 50.51 | 50.51 | 50.51 | 50.51 | 50.51 | 50.51 |
| inert tablets (g) | | | | | | |
| Corn oil loaded | 101 | 38.7 | 57.7 | — | 52.8 | 105 |
| into tablets (g) | | | | | | |
| Total weight (g) | 151 | 89.2 | 108 | 50.51 | 103 | 156 |
| (oil loaded | | | | | | |
| tablets) | | | | | | |
| Tablet weight | 274 | 458 | 458 | 458 | 458 | 275 |
| (mg) | | | | | | |
| Release, Day 1 | 84% | 66% | 75% | 95 mg | 88% | 90% |
| Release, Day 7 | 34% | 65% | 74% | n.a. | 78% | 84% |

Comp. 4C: Tablet edges disintegrated during release test.

### Example 5

### Loadable Tablets of the Invention in Combination with Croscarmellose Sodium (Disintegrant)

Tablet compositions according to the table below were prepared and loaded with corn oil as described in Example 1 (Comp. 5A as comp. 1C, croscarmellose mixed in before MgSt mixing); Example 2 (Comp. 5B, 5D and 5E, croscarmellose mixed in before MgSt mixing); Example 3 (Comp. 5C, croscarmellose mixed in before MgSt mixing); Example 4 (Comp. 5F as comp. 4B, croscarmellose mixed in before MgSt mixing).

Disintegration test: The test was performed in 900 mL de-mineralized water in a USP (United States Pharmacopeia) 701 apparatus at Day 1, Day 7 (1 week) and Day 14 (2 weeks); the results are average, n=6.

The tablet compositions and the release results are shown in Table 5:

TABLE 5

| Composition | Ref2 | 5A | 5B | 5C | 5D | 5E | 5F |
|---|---|---|---|---|---|---|---|
| Mg aluminometasilicate (g) | 23.1 | 18 | 13.6 | 16.2 | 15.5 | 16.5 | 24 |
| Mg stearate (g) | 0.3 | 0.38 | 0.2 | 0.38 | 0.24 | 0.21 | 0.45 |
| PEG6000 (g) | — | 12 | — | 12 | — | — | — |
| KH₂PO₄ (g) | — | — | 1.8 | 1.8 | 2.1 | 2.2 | — |
| Poloxamer 188 (g) | — | — | — | — | — | — | 16 |
| Croscarmellose sodium (g) | 6.61 | 7.59 | 4.4 | 7.59 | 2.25 | 1.14 | 5 |
| Total weight of inert tablets (g) | 30 | 38 | 20 | 38 | 20 | 20 | — |
| Corn oil loaded into tablets (g) | 52.3 | 33.3 | 31.5 | 33.1 | 37.5 | 36.7 | 42.6 |
| Total weight (g) | 82.3 | 71.6 | 51.5 | 71.1 | 57.5 | 56.7 | 88.1 |
| (oil loaded tablets) | | | | | | | |
| Tablet weight (mg) | 275 | 458 | 275 | 458 | 275 | 275 | 458 |
| Disintegration Day 1 (min:sec) | 1:25 | 1:53 | 0:45 | 2:09 | 0:48 | 1:24 | 3:47 |
| Disintegration Day 7 (min:sec) | >78 min | 2:15 | 1:35 | 2:54 | 1:48 | 1:38 | 4:33 |
| Disintegration Day 14 (min:sec) | >180 min | 2:55 | 2:59 | 1:48 | 3:40 | 3:45 | 4:48 |

### Example 6

### Loadable Tablets of the Invention—Loading and Release of EPA/DHA Ethyl Ester Mixture (a Typical Oil Blend Extracted from Fish)

Tablet compositions (inert tablets) according to Table 6 were prepared as described in Example 5 (Comp. 6A as comp. 5B; comp. 6B as comp. 5A; comp. 6C as comp. 5C; comp. 6D as comp. 5F).

For each of the inert tablet compositions, 250 single tablets were loaded with EPA/DHA (fish oil) ethyl ester in a rotating pan to about 95% of their capacity. Rotation was carried out until tablets were free-flowing with dry surfaces. Loading was performed under inert gas to prevent oxidation. The tablets were then stored in an alu-bag purged with inert gas.

The disintegration test was carried out as described in Example 5 at Day 1, Day 7, Day 14 (2 weeks), 4 weeks, 8 weeks; the results are shown for last tablet; (n=6).

The tablet compositions and the release results are shown in Table 6:

TABLE 6

| Composition | Ref3 | 6A | 6B | 6C | 6D |
|---|---|---|---|---|---|
| Mg aluminometasilicate (g) | 133.5 | 56.3 | 52.9 | 50.2 | 52.9 |
| Mg stearate (g) | 1.5 | 0.4 | 1.12 | 1.13 | 1.12 |
| PEG6000 (g) | — | — | 35.3 | 35.8 | — |
| KH₂PO₄ (g) | — | 4.0 | — | 3.5 | — |
| Poloxamer 188 (g) | — | — | — | — | 35.3 |
| Croscarmellose sodium (g) | 15.0 | 15.2 | 22.3 | 22.7 | 22.3 |
| Total weight of inert | 150 | 75.9 | 111.5 | 113.4 | 111.6 |
| tablets (g) | | | | | |
| EPA/DHA oil loaded into | 300 | 113.6 | 97.5 | 97.5 | 97.8 |
| tablets (g) | | | | | |
| Total weight (g) | 450 | 189.7 | 209.0 | 210.9 | 209.4 |
| (oil loaded tablets) | | | | | |
| Tablet weight (mg) | 275 | 305 | 447 | 452.6 | 462 |
| Disintegration Day 1 | 3:24 | 0:30 | 2:00 | 2:20 | 2:00 |
| (min:sec) | | | | | |
| Disintegration Day 7 | 440:02 | 1:24 | 2:10 | 1:16 | 2:12 |
| (min:sec) | | | | | |
| Disintegration Day 14 | >150:00 | 1:26 | 2:42 | 2:12 | 2:36 |
| (min:sec) | | | | | |
| Disintegration 4 weeks | n.a. | 0:54 | 2:16 | 2:40 | 2:58 |
| (min:sec) | | | | | |
| Disintegration 8 weeks | n.a. | 2:56 | 2:50 | 2:30 | 2:40 |
| (min:sec) | | | | | |

### Release Experiment:

Release testing was performed in a USP dissolution apparatus in 900 ml phosphate buffer, pH=6.8 and 100 ml octanol on top to take up the released oil. Tests were performed at 75 rpm and samples were taken from the octanol layer. Released oil was determined by UV absorbance at wavelength 275 nm. The dissolution test was performed at Day 7 (1 week; storage at 25° C.), the results are average (n=3) and shown below:

| Composition | 15 min | 30 min | 45 min | 60 min | 120 min | 180 min |
|---|---|---|---|---|---|---|
| Ref 3* | 1.5% | 1.5% | 2.3% | 3.2% | 36.5% | 86.2% |
| Comp. 6C | 87.5% | 87.5% | 90.3% | 87.9% | 100.3% | NA |

*Results are adjusted to align tablet content

### Example 7

### Loadable Tablets of the Invention—Loading and Release of EPA/DHA Ethyl Ester Mixture (a Typical Oil Blend Extracted from Fish)

Tablet compositions (inert tablets) according to Table 7 were prepared as described in Example 5 (Comp. 7A and 7B as comp. 5A; comp. 7B as comp. 5A; comp. 7C as comp. 5C; comp. 7D as comp. 5F). Comp. 7C, 7D, 7E and 7F were prepared by spraying a suitable KH₂PO₄ solution upon Mg Al metasilicate (Neusilin US2) in a fluid bed for 2 min and leaving it without fluidization for 15 minutes. Then the water was dried away using 80° C. hot fluidization air until constant weight. PEG6000 was heated to 75° C. and sprayed onto the mix of silicate/KH₂PO₄ fluidized in a bed in 3 minutes to provide granules for further processing into tablets as described above.

The tablets were submerged into EPA/DHA (fish oil) ethyl ester until fully loaded with oil. The tablets were then dried with paper and stored in an Alu-bag purged with inert gas.

The disintegration test was carried out as described in Example 5 at Day 1, Day 7, Day 14 (2 weeks), 4 weeks, 8 weeks; the results are shown for last tablet; (n=6).

The tablet compositions and the release results are shown in Table 7:

TABLE 7

| Composition | Ref3 | 7A | 7B | 7C | 7D | 7E | 7F |
|---|---|---|---|---|---|---|---|
| Mg aluminometasilicate (g) | 23.1 | 36.0 | 32.0 | 42.0 | 28.9 | 32.0 | 28.9 |
| Mg stearate (g) | 0.3 | 0.52 | 0.52 | 0.53 | 0.63 | 0.43 | 0.45 |
| PEG6000 (g) | — | 4.0 | 8.0 | 5.1 | 19.2 | 3.6 | 7.2 |
| KH₂PO₄ (g) | — | — | — | 2.9 | 1.9 | 4.4 | 3.9 |
| Croscarmellose sodium (g) | 6.61 | 10.13 | 10.13 | 2.66 | 12.66 | 2.13 | 4.5 |
| Total weight of inert tablets (g) | 30 | 50.65 | 50.65 | 53.19 | 63.29 | 42.55 | 45 |
| EPA/DHA oil loaded (g) | 52.3 | 68.0 | 68.0 | 46.5 | 55.3 | 60.0 | 55 |
| Total weight (g) | 82.3 | 118.7 | 118.7 | 99.7 | 118.6 | 102.6 | 100 |
| (oil loaded tablets) | | | | | | | |
| Tablet weight (mg) | 275 | 371.5 | 371.5 | 572 | 572 | 356 | 410 |
| Disintegration Day 1 (min:sec) | 3:24 | 1:52 | 1:34 | 2:02 | 3:20 | 1:16 | 1:10 |
| Disintegration Day 7 (min:sec) | 40:02 | 1:58 | 2:38 | 2:18 | 4:44 | 1:26 | 1:02 |
| Disintegration Day 14 (min:sec) | 150:0 | 2:48 | 3:14 | 2:22 | 2:12 | 1:32 | 0:56 |
| Disintegr. 4 weeks (min:sec) | n.a. | 4:08 | 3:20 | 3:10 | 2:16 | 1:52 | 1:40 |
| Disintegr. 8 weeks (min:sec) | n.a. | 11:30 | 6:44 | 2:16 | 3:50 | 2:24 | 1:14 |

### Example 8

### Loadable Tablets of the Invention—Loading and Release of Viscoleo MCT Oil

Tablet compositions (inert tablets) 8B and 8C according to Table 8 were prepared as described in Example 7 (comp. 7C). Composition 8A was prepared as described in Example 6 (comp. 6C).

The tablets were loaded by submerging into MCT oil until fully loaded with oil. The tablets were then dried with paper and stored in an HDPE bottle.

The disintegration test was carried out as described in Example 5 at Day 1, Day 7, Day 14 (2 weeks), 4 weeks, 3 months; the results are shown for last tablet; (n=6).

The tablet compositions and the release results are shown in Table 8:

TABLE 8

| Composition | Ref4 | Ref5 | 8A | 8B | 8C |
|---|---|---|---|---|---|
| Mg aluminometasilicate (g) | 133.5 | 118.5 | 76.3 | 75.9 | 28.9 |
| Mg stearate (g) | 1.5 | 1.5 | 1.7 | 1.12 | 0.45 |
| PEG6000 (g) | — | — | 54.4 | 19.0 | 7.2 |
| KH₂PO₄ (g) | — | — | 5.3 | 5.1 | 3.9 |
| Croscarmellose sodium (g) | 15.0 | 30.0 | 34.4 | 11.24 | 4.5 |
| Total weight of inert tablets (g) | 150 | 150 | 172.1 | 112.4 | 45 |
| Viscoleo MCToil loaded into | 197 | 186 | 154.0 | 144.0 | 55 |
| tablets (g) | | | | | |
| Total weight (g) | 347 | 336 | 326.1 | 256.4 | 100 |
| (oil loaded tablets) | | | | | |
| Tablet weight (mg) | 137 | 137 | 556.5 | 390 | 410 |
| Disintegration Day 1 (min:sec) | 0:30 | 0:24 | 3:20 | 1:10 | 1:32 |
| Disintegration Day 7 (min:sec) | — | — | 1:36 | 1:30 | 1:02 |
| Disintegration Day 14(min:sec) | 20:00 | 10:00 | 2:00 | 1:14 | 1:18 |
| Disintegr. 4 weeks (min:sec) | . | — | 2:20 | 1:28 | 1:26 |
| Disintegr. 3 months (min:sec) | 44:00 | 19:00 | 3:52 | 2:46 | 1:26 |

1. (canceled)

2. A loadable solid porous composition consisting essentially of porous silicium dioxide (silicon dioxide) pre-deposited with a release enhancing agent.

3. The composition of claim 2, wherein the composition is a compressed or molded tablet having a hardness of from 20 N to about 150 N.

4. The composition of claim 2, wherein the composition comprises a granulate.

5. The composition of claim 2, wherein said composition has a porosity of at least 30% v/v.

6. The composition of claim 2, wherein the porous silicium dioxide is present in a concentration of about 20% w/w or more in the unloaded composition.

7. The composition of claim 2, wherein the porous silicium dioxide is selected from magnesium aluminum metasilicate, magnesium aluminum silicate, aluminium metasilicate, and mixtures thereof.

8. The composition of claim 2, wherein the release enhancing agent is present in a concentration of about 2% w/w to 70% w/w (based on the total weight of the composition before loading).

9. The composition of claim 2, wherein the release enhancing agent is selected from a polymer, an inorganic salt or an inorganic aqueous hydrogen phosphate, and mixtures thereof.

10. The composition of claim 9, wherein the release enhancing agent is a polymer selected from a polyethylene glycol, a poloxamer, a polyethylene oxide, an alkyl cellulose, e.g. HPMC, or polyvinyl alcohol (PVA), polyvinyl acetate phthalate, polyvinyl acetate and mixtures thereof.

11. The composition of claim 10, wherein the polymer is a polyethylene glycol having an average molecular weight in a range of from about 400 to about 100,000 and mixtures thereof; or the polymer is a poloxamer or other block copolymers of ethylene oxide and propylene oxide, and mixtures thereof, having an average molecular weight in a range of from about 400 to about 100,000; and mixtures thereof.

12. The composition of claim 9, wherein the release enhancing agent is an inorganic salt or is an inorganic aqueous hydrogen phosphate.

13. The composition of claim 12, wherein the release enhancing agent is potassium dihydrogen phosphate (KH₂PO₄) in a concentration from 0.1 M to 10 M.

14. The composition of claim 2, wherein the release enhancing agent is selected from a mixture of a polymer and an inorganic salt, from a mixture of polymers, or from a mixture of salts.

15. The composition of claim 2 further comprising a disintegrant.

16. The composition of claim 15, wherein the disintegrant is selected from croscarmellose sodium, alginic acid or alginates, microcrystalline cellulose, hydroxypropyl cellulose and other cellulose derivatives, crospovidone, polacrillin potassium, sodium starch glycolate, starch, pregelatinized starch, and carboxymethyl starch.

17. The composition of claim 15, wherein the concentration of disintegrant is from 1% w/w to 20% w/w, based on the total weight of the composition before loading.

18. The composition of claim 2, wherein the composition is in the form of a compressed tablet, and the release enhancing agent is uniformly distributed in the tablet.

19. The composition of claim 2, wherein the composition is selected from a pharmaceutically or nutritionally acceptable composition.

20. A solid pharmaceutical composition comprising:
(a) a porous silicium dioxide pre-deposited with a release enhancing agent; and
(b) a pharmaceutically acceptable oily substance and/or a pharmaceutically active substance loaded in the porous silicium dioxide.

21. The composition of claim 20, wherein the porous silicium dioxide has a particle size between 44 micron and 177 micron prior to pre-deposition with the release enhancing agent.

22. The composition of claim 20, wherein the pharmaceutically active substance or pharmaceutically acceptable oily substance is present in a concentration of about 5% w/w or more (based on the total weight of the composition after loading).

23. The composition of claim 20, wherein the pharmaceutically active substance or pharmaceutically acceptable oily substance comprises one or more structured triglycerides, mono-glycerides, fatty acids, or esters of fatty acids, or mixtures thereof.

24. The composition of claim 20, wherein the pharmaceutically active substance or pharmaceutically acceptable oily substance is selected from apricot oil, almond oil, avocado oil, castor oil, coconut fat, cocoa butter, corn oil, cotton seed oil, grape seed oil, jojoba oil, linseed oil, maize oil, olive oil, palm oil, peanut oil, poppy seed oil, rape seed oil, sesame oil, soybeen oil, sunflower oil, thistle seed oil, walnut oil, wheat germ oil, hydrogenated peanut oil, hydrogenated palm kernels oil, hydrogenated cottonseed oil, hydrogenated soya oil, hydrogenated castor oil, hydrogenated coconut oil, beef tallow, lard, tall oil, whale oil, fish oil, cod liver oil, flaxseed oil, palm kernel oil, safflower oil, shea nut oil and mixtures thereof, as well as structured triglycerides, mono-glycerides, fatty acids, or esters of fatty acids from said oils, or mixtures thereof.

25. The composition of claim 20, wherein the pharmaceutically active substance or pharmaceutically acceptable oily substance has a melting point of at least about minus (−) 30° C. and at the most about 100° C.

26. The composition of claim 20, wherein the pharmaceutically active substance or pharmaceutically acceptable oily substance is released from the composition upon contact with an aqueous environment in an amount of at least 60% w/w after storage for one week.

27. The composition of claim 20, wherein the composition is in the form of a compressed tablet, and the release enhancing agent is uniformly distributed in the tablet.

28. The composition of claim 20, wherein the composition is prepared by
(a) obtaining a granulate consisting essentially of the porous silicium dioxide and the release enhancing agent, and
(b) loading the pharmaceutically active substance or pharmaceutically acceptable oily substance into the granules, until the pharmaceutically active substance or pharmaceutically acceptable oily substance is loaded to about 70% or more of the loading capacity.

29-31. (canceled)

32. A method for the preparation of solid porous granulate comprising a porous silicium dioxide, a release enhancing agent and a pharmaceutically acceptable oily substance, comprising the steps of:
i) preparing a granulate of the porous silicium dioxide and the release enhancing agent,
ii) loading the pharmaceutically acceptable oily substance into the granules, until the pharmaceutically acceptable oily substance is loaded to about 70% or more of the loading capacity,
iii) providing the solid porous granulate.

33. The method of claim 32, further comprising the step of:
iv) filling the solid porous granulate into capsules.

34. A method for the preparation of a loadable solid porous tablet comprising porous silicium dioxide and a release enhancing agent, and optionally a disintegrant, the method comprising the steps of:
i) preparing a granulate of the porous silicium dioxide and the release enhancing agent, and optionally a disintegrant,
ii) providing the loadable solid porous granulate,
iii) compressing the granulate into loadable tablets,
or, alternatively,
a) preparing a granulate of the porous silicium dioxide, and optionally a disintegrant,
b) compressing the granulate into loadable tablets,
c) uniformly distributing the release enhancing agent into the tablet by force,
d) providing the loadable tablets.

35. A method for the preparation of a solid porous tablet comprising a porous silicium dioxide, a release enhancing agent, optionally a disintegrant, and a pharmaceutically acceptable oily substance, the method comprising the steps of:
i) preparing a loadable tablet comprising a porous silicium dioxide, a release enhancing agent, optionally a disintegrant, and a pharmaceutically acceptable oily substance, and
ii) loading the pharmaceutically acceptable oily substance into the tablet until the pharmaceutically acceptable oily substance is loaded to about 70% or more of the loading capacity.

36. The method of claim 35, wherein the loading is performed by placing the tablet in an excess amount of the pharmaceutically acceptable oily substance for a sufficient amount of time.

37. The method of claim 36, wherein the loading is performed under pressure.

38. The method of claim 36, wherein the time period of loading the pharmaceutically acceptable oily substance is from 30 min to 3 hours.

39. (canceled)

40. A solid porous granulate comprising a porous silicium dioxide, a release enhancing agent and a pharmaceutically active substance, obtainable by the method of claim 32.

41. A capsule comprising solid porous granulate comprising a porous silicium dioxide, a release enhancing agent and a pharmaceutically active substance, obtainable by the method of claim 33.

42. A loadable solid porous tablet comprising a porous silicium dioxide and a release enhancing agent, and optionally a disintegrant, obtainable by the method of claim 34.

43. A solid porous tablet comprising a porous silicium dioxide, a release enhancing agent, optionally a disintegrant, and a pharmaceutically active substance, obtainable by the method of claim 35.

44. (canceled)
